(19) United States
(12) Patent Application Publication
(10) Pub. No.: US 2022/0323306 A1
(43) Pub. Date: Oct. 13, 2022

(54) FREE-FLOWING POWDER FORM CONDITIONER

(71) Applicant: OWA Haircare, Inc., New York, NY (US)

(72) Inventor: Kailey R. Bradt, Big Flats, NY (US)

(21) Appl. No.: 17/711,292

(22) Filed: Apr. 1, 2022

Related U.S. Application Data
(60) Provisional application No. 63/170,220, filed on Apr. 2, 2021.

(51) Int. Cl. A61K 8/02; A61Q 5/12; A61K 8/31; A61K 8/41; A61K 8/34; A61K 8/73; A61K 8/368; A61K 8/58; A61K 8/37

(52) U.S. Cl. CPC A61K 8/022; A61Q 5/12; A61K 8/31; A61K 8/416; A61K 8/342; A61K 8/732; A61K 8/737; A61K 8/368; A61K 8/58; A61K 8/37; A61K 2800/524; A61K 2800/48

(57) ABSTRACT

A free-flowing, powder form conditioner which includes, a one or more conditioning agents; one or more emulsifiers; one or more fatty alcohols; one or more chelating agents; and one or more pH adjusters. A method of using the powder conditioner.

### CROSS-REFERENCE TO RELATED APPLICATIONS

This application claims the benefit of U.S. Provisional Patent Application No. 63/170,220, filed Apr. 2, 2021. The entire contents of this application are incorporated herein by reference in their entirety.

### FIELD OF THE INVENTION

The present invention relates to compositions and methods of making a free-flowing powder conditioner that hydrates upon contact with water. More particularly, the conditioner powder of the present invention, upon contact with water and shear stresses, forms a liquid that does not compromise creamy texture or scent, creating a lightweight and moisturizing conditioner.

### BACKGROUND OF THE INVENTION

Conditioner is conventionally sold as a liquid, which is made up mostly of water, in some cases up to 90% water by weight. Liquid conditioners are inconvenient for travel, and fluids are often restricted on flights. Additionally, liquid conditioners are frequently manufactured by environmentally unsustainable methods and the distribution methods pose environmental risks. For instance, due to the use and waste of plastic from packaging, the use and transportation of fresh water from the manufacturing area, and excessive use of fuel and energy necessary for manufacturing and transportation, conditioner production tends to be environmentally unfriendly. Accordingly, a need exists to promote and maintain an environmentally friendly solution to this industry in which there is a need to reduce waste, increase efficiency, and promote a green supply chain.

### BRIEF SUMMARY OF THE INVENTION

This invention relates to a free-flowing powdered conditioner, which overcomes the shortcomings of the prior art and provides additional advantages. This conditioner powder readily forms a liquid conditioner when combined with water and minimal shear stress, such as the shear stress of rubbing hands together. The resulting liquid conditioner is comparable to that of a traditional liquid conditioner in that it can contain all the features that can be found in a traditional liquid conditioner.

Disclosed is a powdered conditioner that, in one aspect, includes: one or more conditioning agents, an emulsifier, a fatty alcohol, a chelating agent, and a pH buffer, and wherein the powdered conditioner is free-flowing.

In one aspect the invention provides a powdered conditioner comprising: one or more conditioning agents; one or more emulsifiers; one or more fatty alcohols; one or more chelating agents; and one or more pH adjusters, wherein the powdered conditioner is free-flowing.

In some embodiments of the invention, the powdered conditioner of the invention further comprises one or more carrier materials or flow agents.

In other embodiments of the powdered conditioner of the invention, the one or more conditioning agents comprise approximately 4% to approximately 20% by weight of the formulation. In certain embodiments of the powdered conditioner of the invention, the one or more conditioning agents are selected from the group consisting of squalane, ethylhexyl glutamate, cocos nucifera (coconut) oil, argania spinosa kernel oil, behentrimonium chloride, guar hydroxypropyltrimonium chloride, stearamidopropyl dimethylamine, hydrolyzed rice protein, ethyl hexyl olivate, aloe barbadensis leaf juice and combinations thereof. In particular embodiments, the one or more conditioning agents include squalane. In other particular embodiments, the one or more conditioning agents include behentrimonium chloride in an amount of 1.0%-5.0%.

In other embodiments of the powdered conditioner of the invention, the one or more emulsifiers comprise approximately 5% to approximately 20% by weight of the formulation. In certain embodiments, the one or more emulsifiers are selected from the group consisting of sodium stearoyl glutamate, cetearyl glucoside, hydroxypropyl starch phosphate and combinations thereof.

In still other embodiments of the powdered conditioner of the invention, the one or more fatty alcohols comprise approximately 5% to approximately 30% by weight of the formulation. In certain embodiments, the one or more fatty alcohols are selected from the group consisting of cetearyl alcohol, lauryl alcohol, cetyl alcohol, myristyl alcohol, stearyl alcohol, cetearyl alcohol, behenyl alcohol and combinations thereof.

In other embodiments of the powdered conditioner of the invention, the one or more chelating agents comprise approximately 0.1% to approximately 4.0% by weight of the formulation. In certain embodiments, the one or more chelating agents are selected from the group consisting of sodium phytate, disodium EDTA, trisodium ethylenediamine disuccinate, sodium citrate, citric acid and combinations thereof.

In still other embodiments of the powdered conditioner of the invention, the one or more pH adjusters comprise approximately 0.2% to approximately 4.0% by weight of the formulation. In certain embodiments, the one or more pH adjusters agents are selected from the group consisting of citric acid, sodium bicarbonate, sodium hydroxide, L-arginine and combinations thereof. In particular embodiments, the powdered condition of the invention has a pH of 3.5-4.5 when added at 10% to a water solution.

In other embodiments of the powdered conditioner of the invention, the one or more carrier materials or flow agents comprise approximately 50% to approximately 90% by weight of the formulation. In certain embodiments, the one or more carrier materials or flow agents are selected from the group consisting of tapioca starch, corn starch, rice starch, silica, potato starch, arrowroot starch, tricalcium phosphate, powdered cellulose, magnesium stearate, sodium ferrocyanide, potassium ferrocyanide, calcium ferrocyanide, sodium silicate, silicon dioxide, calcium silicate, magnesium trisilicate, talcum powder, sodium aluminosilicate, potassium aluminium silicate, calcium aluminosilicate, bentonite, aluminium silicate, stearic acid, polydimethylsiloxane and combinations thereof.

In some embodiments of the powdered conditioner of the invention, the powdered conditioner of the invention comprises one or more additives. In certain embodiments, the one or more additives, in total, comprise up to approximately 20% by weight of the formulation. In particular embodiments, the one or more additives comprise one or more preservatives, fragrances, thickening agents, or colorants. In specific embodiments, at least one additive is a fragrance wherein the fragrance includes one or more essential oils.

Also disclosed is a method, the method including providing a powdered conditioner, applying an amount of powdered conditioner to a user's hands, rubbing the amount of powdered conditioner between the user's hands water to form a liquid conditioner having a creamy-to-gel texture, applying the liquid conditioner to the user's hair, massaging the liquid conditioner into the user's skin and hair, and rinsing the liquid conditioner out of the user's hair.

### DETAILED DESCRIPTION OF THE INVENTION

The following is a detailed description provided to aid those skilled in the art in practicing the present disclosure. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either of those included limits are also included in the disclosure.

All numerical values within the detailed description and the claims herein are modified by “about” or “approximately” the indicated value, and take into account experimental error and variations that would be expected by a person having ordinary skill in the art.

The following terms are used to describe the present disclosure. Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the term “free-flowing” refers to a powder composition wherein the particles of the composition are free, or substantially free, from clumping, caking, or agglomerating, thereby maintaining a dry particulate formation. A free-flowing powder is distinct from a paste or paste-like composition which experiences significant caking or clumping based on the moisture content. In particular embodiments, the powdered conditioner of the invention is made up of a variety of free-flowing powder components. In other embodiments, the powdered conditioner of the invention is a free-flowing powder.

By “powder form” it is meant that ingredients are, in the finished form a powder and the wet ingredients that make up the powder form do not exceed 20% of the final powder form.

As used herein, the term “plant derived” refers to the origin of the component. For example, as described Philippe et al. “Naturalness: its assessment in the development of sustainable and green chemistry ingredients. An industrial commitment” Annales Des Falsifications, De L′expertise Chimique & Toxicologique N° 985-2ème semestre 2016), “A raw material is considered from plant origin if its percentage in Carbon of plant origin exceeds 50% of its total Carbons. This rating, taking into account the percentage of renewable carbon, is of great interest as an ASTM standard D6866 [18] can measure this rate using radiocarbon (¹⁴C) analysis.”

By “conditioning” it is meant that the hair has improved manageability, and provides a soft feel to the hair after application.

It should also be understood that, in certain methods described herein that include more than one step or act, the order of the steps or acts of the method is not necessarily limited to the order in which the steps or acts of the method are recited unless the context indicates otherwise.

Descriptions of well-known materials, fabrication tools, processing techniques, etc., are omitted so as to not unnecessarily obscure the invention in detail. It should be understood, however, that the detailed description and the specific examples, while indicating embodiments of the invention, are given by way of illustration only, and are not by way of limitation. Various substitutions, modifications, additions and/or arrangements within the spirit and/or scope of the underlying inventive concepts will be apparent to those skilled in the art from this disclosure.

Generally stated, disclosed herein is a free-flowing, powder form conditioner that hydrates upon contact with water. The free-flowing, powder form conditioner of the present invention, upon contact with water and minimal shear stress, forms a liquid that does not compromise creamy texture or scent as compared to traditional liquid conditioners, creating a lightweight and moisturizing conditioner from a dry powder.

Currently, consumers have a large selection of liquid conditioners, however there is an absence of conditioner powder that can achieve the same features as that of a traditional liquid conditioner. The present invention discloses a free-flowing, powder form conditioner option, which, upon contact with water, hydrates to form a traditional liquid conditioner with conditioning properties, creamy texture, and other features, from the moisturizing of the conditioner, as well as through the use of optional additives such as ingredients that reduce frizz and/or detangle, soften, add shine in some embodiments.

According to one or more embodiment, some or all of the ingredients used in the preparation of the conditioner powder of this disclosure should possess good water solubility, meaning that upon contact with water, some or all of the ingredients will be soluble. It has been found that the thickeners used in the preparation of the conditioner powder of this invention should thicken with minimal shear stresses, for example, by a rubbing motion of the hands together manually without external force.

The present invention is concerned with the discovery that combining together components which may include one or more conditioning agents; one or more emulsifiers; one or more fatty alcohols; one or more chelating agents; and one or more pH adjusters, can be formulated into a conditioner powder which is a free-flowing powder, which, upon contact with water and shear stress, forms a liquid conditioner that does not compromise creamy/smooth texture or scent, creating a lightweight and moisturizing conditioner. By moisturizing, the conditioner may be gentle on hair, including but not limited to having a pH of approximately 3.5 to 4.5, and in some embodiments, between approximately 4.0 and 4.5. The conditioning qualities include leaving hair soft, tangle-free and moisturized. By lightweight, the conditioner may be weightless on hair while leaving the hair volumized., including, but not limited to, not adding physical indication of increased weight.

The free flowing powdered conditioner of the invention is formulated in a carrier which comprises one or more carrier materials or flow agents. Such carrier materials and flow agents include, but are not limited to tapioca starch, corn starch, rice starch, silica, potato starch, arrowroot starch, tricalcium phosphate, powdered cellulose, magnesium stearate, sodium bicarbonate, sodium ferrocyanide, potassium ferrocyanide, calcium ferrocyanide, bone phosphate, sodium silicate, silicon dioxide, calcium silicate, magnesium trisilicate, talcum powder, sodium aluminosilicate, potassium aluminum silicate, calcium aluminosilicate, bentonite, aluminum silicate, stearic acid, polydimethylsiloxane. In certain embodiments, the carrier is present in an amount of 50-90% by weight of the total free flowing powdered conditioner. In other embodiments, the carrier is present in an amount of 55-85% by weight of the total free flowing powdered conditioner. In still other embodiments, the carrier is present in an amount of 60-80% by weight of the total free flowing powdered conditioner.

The free flowing powdered conditioner of the invention comprises at least one conditioning agent. Conditioning agents and/or slip agents may be useful in the practice of the present invention for increasing the wellness aspects of powdered conditioners according to some embodiments. For instance, conditioning agents and/or slipping agents may include conditioning agents in which good water solubility is possessed, including but not limited to plant derived proteins, plant extracts, and quaterniums. In some embodiments, the conditioning agents may include, but are not limited to, squalane, ethylhexyl glutamate, cocos nucifera (coconut) oil, argania spinosa kernel oil, behentrimonium chloride, guar hydroxypropyltrimonium chloride, stearamidopropyl dimethylamine, hydrolyzed rice protein, ethyl hexyl olivate, aloe barbadensis leaf juice or a combination thereof. In particular embodiments, the conditioning agent is squalane. In particular embodiments, the conditioning agent is behentrimonium chloride.

The total amount of conditioning and/or slipping agent necessary to obtain the desired effect may include from about 4.0% to 20% by weight. In some embodiments, the total amount of conditioning agent is from about 4.0% to about 10% by weight or 20% to about 30% by weight. In some embodiments, the amount of conditioning agent, which may include a slip agent, can include approximately 4%, approximately 10%, approximately 15%, approximately 20%, approximately 25%, approximately 30%. Additionally, a plurality of conditioning agents may be used, with or without a separate slip agent. In still particular embodiments, when the conditioning agent is behentrimonium chloride, the amount of conditioning agent is not above 5.0%.

The free flowing powdered conditioner of the invention comprises at least one emulsifying agent. In some embodiments, the emulsifying agents may include, but are not limited to, sodium stearoyl glutamate, cetearyl glucoside, hydroxypropyl starch phosphate.

The total amount of emulsifying agent necessary to obtain the desired effect may include from about 5.0% to 20% by weight. In some embodiments, the amount of emulsifying agent can include approximately 2.5%, approximately 3.0%, approximately 3.5%, approximately 4.0%, approximately 4.5%, approximately 7.5%, approximately 10%, approximately 12.5%, approximately 15%, approximately 17.5%, or approximately 20%. Additionally, a plurality of emulsifying agents may be used.

The free flowing powdered conditioner of the invention comprises at least one fatty alcohol. In some embodiments, the fatty alcohols may include, but are not limited to, cetearyl alcohol, lauryl alcohol, cetyl alcohol, myristyl alcohol, stearyl alcohol, cetearyl alcohol, behenyl alcohol.

The total amount of fatty alcohol necessary to obtain the desired effect may include from about 5.0% to 30% by weight. In some embodiments, the amount of emulsifying agent can include approximately 5.0%, approximately 10%, approximately 15%, approximately 20%, approximately 25%, approximately or approximately 30%. Additionally, a plurality of fatty alcohols may be used.

The free flowing powdered conditioner of the invention comprises at least one chelating agent. In some embodiments, one or more chelating agents can be utilized, which can include, for example, sodium phytate, disodium EDTA, trisodium ethylenediamine disuccinate, sodium citrate, citric acid.

The total amount of chelating agent necessary to obtain the desired effect may include from about 0.1% to 4.0% by weight. In some embodiments, the amount of chelating agent can include approximately 0.1%, approximately 0.2%, approximately 0.3%, approximately 0.4%, approximately 0.5%, approximately 1.0%, approximately 2.0%, approximately 3.0%, or approximately 4.0%. Additionally, a plurality of chelating agents may be used.

The free flowing powdered conditioner of the invention comprises at least one pH adjuster or pH buffer. In some embodiments, one or more pH adjusters can be utilized, which can include, for example, citric acid, sodium bicarbonate, sodium hydroxide, and L-arginine. In particular embodiments, the pH adjuster is citric acid. In certain embodiments, the pH of the powder conditioner is between about 3.5 to about 4.5 when measured at a concentration of 10% in water.

The total amount of pH adjuster necessary to obtain the desired pH may include from about 0.2% to 4.0% by weight. In some embodiments, the amount of pH adjuster can include approximately 0.2%, approximately 1.0%, approximately 1.5%, approximately 2.0%, approximately 2.5%, approximately 3.0%, approximately 3.5%, or approximately 4.0%. Additionally, a plurality of pH adjusters may be used.

Thickening agents which are useful in the practice of the present invention include thickeners in which good water solubility is possessed and minimal shear stresses from rubbing hands can thicken the conditioner powder and water mixture to obtain the desirable viscosity of the liquid conditioner including, but not limited to, hydroxypropyl starch phosphate, cellulose gum, xanthan gum, tapioca starch, and guar gum. The amount of thickening agent, such as cellulose gum, to obtain the desired effect may include from approximately 5% to approximately 30% by weight. The water solubility of the thickeners, as well as the minimal shear stress require to activate thickeners, aids in the ability to form a liquid conditioner having a creamy or smooth texture, equivalent to that of liquid conditioners. It is understood that one or a plurality of thickening agents may be utilized. The desired viscosity of the hydrated conditioner can be between approximately 1,000 cP and approximately 10,000 cP as measured at 10 rpm at room temperature. In some embodiments, the viscosity may be approximately 2,000 cP, approximately 3,000 cP approximately 4,000 cP, approximately 5,000 cP approximately 6,000 cP, approximately 7,000 cP approximately 8,000 cP, or approximately 9,000 cP.

The free-flowing, powder form conditioner, according to some embodiments of the present invention, may further include one or more optional additives. Additives can include, for instance, preservatives, fragrances, and colorants. When these additives are present in the free-flowing conditioner powder, they may range from 0.1% to 30% by weight. In some embodiments, the amount of total additives can include approximately 1%, approximately 5%, approximately 10%, approximately 15%, approximately 20%, or approximately 25%. In some embodiments, preservatives can be utilized, which may include any commercially available preservative, including but not limited to sodium benzoate, sodium dehydroacetate, and preservatives. Additionally, fragrances may be utilized, which may include any naturally-derived fragrances, including but not limited to essential oils. In some embodiments, one or more colorants may be utilized, which can include rose hips, clays, and tea leaves, among other natural colorants.

In some embodiments, the additives may not be supplied in a dry form like the other ingredients, so long as the final conditioner powder is dry following the addition of the optional ingredients. The dry ingredients may be capable of absorbing any moisture and still resulting in a completely or substantially dry conditioner powder, or the solvent of the additive may be capable of evaporating during manufacturing and/or mixing of the powdered conditioner. Substantially dry can include less than approximately 30% moisture, inclusive of water, in some embodiments, and in further embodiments can include less than 20%, less than 10%, less than 5%, less than 1%, or completely dry in some embodiments. With particular regard to water content, the free flowing powdered conditioner of the invention can include less than approximately 20% water in some embodiments, and in further embodiments can include less than 15%, less than 10%, less than 5%, less than 1%.

The powdered conditioner of the invention can be prepared, generally, by combining the free flowing powders together in a suitable container to create a powder mixture of said ingredients. The resultant powdered conditioner is also a free flowing powder. Generally, as used throughout, powder can refer to any bulk solid, fine or coarse, including but not limited to dust, granules, and granulations. The term free flowing refers to the flowability of the powder, specifically to its ability to flow in the processing steps required to mix or use the powdered conditioner.

Non-free flowing powders can be added to the mixture so long as the final product maintains the appearance and properties of the described present invention. Non-free flowing powders can include essential oils and solids ingredients in a solvent such as glycerin or an alcohol and other solids that can provide exfoliating properties and do not exhibit water solubility. The final mixture can be obtained by any appropriate methods. In some embodiments, a milling machine may be utilized for preparing powders, as well as for mixing the ingredients. A ribbon blender may be utilized for mixing the ingredients. The blended mixture may be passed through a sieve.

Once mixed, the powdered conditioner is completely or nearly completely dry. The powdered conditioner is capable of exhibiting a smooth or creamy liquid texture with substantially no clumps or lumps and can function as a liquid conditioner when combined with water and shear stress. The water need not be any specific temperature to form the liquid conditioner in the hands, that is the powder will work with hot water, cold water, or any combination thereof. Water may be added to an amount of powdered conditioner in approximately 0.1 gram to approximately 1.0 gram of dry conditioner with any water desired. The water can include only what is present on a user's wet hands with no additional water, in some embodiments. It should be noted that the water can be added to the powdered conditioner directly in the palms of one's hands, in some embodiments, or by adding water to the conditioner in the hair, in other embodiments. The powdered conditioner emulsifies and is capable of becoming a cream-like to gel-like liquid without agitation by shaking or being otherwise mixed together in a separate container, without centrifugation or other processes where external shear forces are needed to create the creaminess or forming a liquid conditioner. That is, the shear stress, or shear force, provided by rubbing hands together, manually, is all that is required for creating a useable liquid conditioner, with no need to premix any further ingredients, nor requiring any extra containers for shaking or mixing ingredients or solvents. The shear stress provided by the rubbing motion of hands is sufficient to provide adequate creaminess and mixing the ingredients together to provide the scent from any fragrance which may have been added, and mixing any colorants sufficiently.

In some embodiments, the powdered conditioner is free of certain ingredients which are considered less sustainable or which are harmful. For instance, the powdered conditioner can be free of artificial colors, artificial fragrances, silicones, phthalates, animal-derived products, including any raw material of animal origin, parabens, and ingredients which have been tested on animals.

In some embodiments, the powdered conditioner can be analyzed for naturalness, described as naturally-derived, using an index combining the origin and denaturation of the raw materials. For instance, an origin index, similar to that developed by L'Oreal®, has a scale of 1 to 5. Any animal origin material receives a score of 1, fossil based material a score of 2, any mineral based material earns a score of 3, farmed plant material gets a score of 4, and organic or wild plant material gets a score of 5. Raw material of plant origin includes any material for which its percentage of carbon being plant origin exceeds 50% of total carbons. Such a rating takes into account renewable carbon and can be analyzed using 14C radiocarbon analysis. Thus, materials averaging over a threshold can be considered of desirable origin. In some embodiments, powdered conditioner according to the embodiments disclosed above, can include an origin index of 3.8 or higher, and in some embodiments, may include an origin index of 4.0, 4.2, 4.4, or 4.6.

Disclosed herein is also a method, the method including methods of using a powdered conditioner as disclosed above. The method, in some embodiments, includes providing a powdered conditioner according to any of the above embodiments, applying an amount of powdered conditioner to a user's hands. The amount of conditioner can include the specified weights disclosed above. The powdered conditioner is then rubbed between the user's hands, in some embodiments, just in wet hands. In some embodiments, the conditioner powder is rubbed between the hands under water, with an amount of water, or under running water, in order to form a liquid conditioner having a smooth or creamy or gel-like texture with substantially no lumps or clumps.

The liquid conditioner is then applied to the user's hair and massaged into the user's skin and hair for a period of time consisting of between approximately 20 seconds and approximately 10 minutes. The period of time can include, for instance, between approximately 30 seconds and 5 minutes, and approximately 1 minute and 2 minutes. In some embodiments, more water may be added to the conditioner by adding water to the hair with the conditioner already applied, allowing for control over the texture before and during use of the conditioner. The conditioner is then rinsed out of the user's hair.

### EXAMPLES

In a particular embodiment, the invention provides a free-flowing powdered conditioner comprising the following formulation:

TABLE 1
Formulation (General)

| Common Name | Quantity by weight |
|---|---|
| Carrier Material(s)/Flow Agent(s) | 50.0-90.0% |
| Conditioning agent(s) | 4.0-20.0% |
| Emulsifier(s) | 5.0-20.0% |
| Fatty Alcohol(s) | 5.0-30.0% |
| Chelating Agent(s) | 0.1-4.0% |
| pH Adjuster/pH Buffer | 0.2-4.0% |
| Preservative(s) | 0.0-1.0% |
| Fragrance(s) | 0.0-5.0% |
| Total | 100% |

Particular formulations may include other ingredients such as thickeners, fragrances and colorants. Exemplary formulations include.

TABLE 2
Formulation 1

| Component | Quantity by weight |
|---|---|
| Corn Starch | 65.60% |
| Guar Hydroxypropyltrimonium Chloride | 8.00% |
| Cocos Nucifera (Coconut) Oil | 2.00% |
| Hydroxypropyl Starch Phosphate | 4.00% |
| Sodium Stearoyl Glutamate | 15.00% |
| Cetearyl Alcohol | 3.00% |
| Sodium Phytate | 0.20% |
| Sodium Benzoate | 0.20% |
| Citric Acid Anhydrous | 1.50% |
| Fragrances and other additives | 0.5% |
| Total | 100% |

The resultant powdered conditioner is a free-flowing powder and substantially dry.

TABLE 3
Formulation 2

| Component | Quantity by weight |
|---|---|
| Tapioca Starch | 55.00% |
| Guar Hydroxypropyltrimonium Chloride | 14.00% |
| Aloe Barbadensis Leaf Juice | 0.50% |
| Argania Spinosa Kernel Oil | 1.00% |
| Squalane | 0.70% |
| Ethyl Hexyl Olivate | 7.00% |
| Sodium Stearoyl Glutamate | 10.00% |
| Behentrimonium Chloride | 4.00% |
| Cetyl Alcohol | 1.00% |
| Sodium Phytate | 0.70% |
| Fragrances and other additives | 2.10% |
| Citric Acid Anhydrous | 4.00% |
| Total | 100% |

The resultant powdered conditioner is a free-flowing powder and substantially dry.

TABLE 4
Formulation 3

| Component | Quantity by weight |
|---|---|
| Tapioca Starch | 68.35% |
| Guar Hydroxypropyltrimonium Chloride | 7.00% |
| Squalane | 0.25% |
| Ethylhexyl Olivate | 5.00% |
| Sodium Stearoyl Glutamate | 15.00% |
| Behentrimonium Chloride | 2.00% |
| Cetearyl Alcohol | 0.70% |
| Stearyl Alcohol | 0.30% |
| Sodium Phytate | 0.20% |
| Sodium Benzoate | 0.20% |
| Citric Acid Anhydrous | 1.00% |
| Total | 100% |

The resultant powdered conditioner is a free-flowing powder and substantially dry.

### Incorporation by Reference

The entire contents of all patents, published patent applications and other references cited herein are hereby expressly incorporated herein in their entireties by reference.

### Equivalents

Those skilled in the art will recognize, or be able to ascertain using no more than routine experimentation, many equivalents to the specific embodiments and methods described herein. Such equivalents are intended to be encompassed by the scope of the following claims.

It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

What is claimed is:

1. A powdered conditioner comprising:
one or more conditioning agents;
one or more emulsifiers;
one or more fatty alcohols;
one or more chelating agents;
and one or more pH adjusters,
wherein the powdered conditioner is free-flowing.

2. The powdered conditioner of claim 1, further comprising one or more carrier materials or flow agents.

3. The powdered conditioner of claim 1, wherein the one or more conditioning agents comprise approximately 4% to approximately 20% by weight of the formulation.

4. The powdered conditioner of claim 1, wherein the one or more conditioning agents are selected from the group consisting of squalane, ethylhexyl glutamate, cocos nucifera (coconut) Oil, argania spinosa kernel oil, behentrimonium chloride, caprylyl glycol, guar hydroxypropyltrimonium chloride, stearamidopropyl dimethylamine, hydrolyzed rice protein, ethyl hexyl olivate, aloe barbadensis leaf juice and combinations thereof.

5. The powdered conditioner of claim 1, wherein the one or more conditioning agents include squalane.

6. The powdered conditioner of claim 1, wherein the one or more conditioning agents include behentrimonium chloride in an amount of 1.0%-5.0%.

7. The powdered conditioner of claim 1, wherein the one or more emulsifiers comprise approximately 5% to approximately 20% by weight of the formulation.

8. The powdered conditioner of claim 1, wherein the one or more emulsifiers are selected from the group consisting of sodium stearoyl glutamate, cetearyl glucoside, hydroxypropyl starch phosphate, glyceryl laurate, glyceryl undecylenate and combinations thereof.

9. The powdered conditioner of claim 1, wherein the one or more fatty alcohols comprise approximately 5% to approximately 30% by weight of the formulation.

10. The powdered conditioner of claim 1, wherein the one or more fatty alcohols are selected from the group consisting of cetearyl alcohol, lauryl alcohol, cetyl alcohol, myristyl alcohol, stearyl alcohol, behenyl alcohol and combinations thereof.

11. The powdered conditioner of claim 1, wherein the one or more chelating agents comprise approximately 0.1% to approximately 4.0% by weight of the formulation.

12. The powdered conditioner of claim 1, wherein the one or more chelating agents are selected from the group consisting of Sodium Phytate, Disodium EDTA, trisodium ethylenediamine disuccinate, sodium citrate, citric acid and combinations thereof.

13. The powdered conditioner of claim 1, wherein the one or more pH adjusters comprise approximately 0.2% to approximately 4.0% by weight of the formulation.

14. The powdered conditioner of claim 1, wherein the one or more pH adjusters agents are selected from the group consisting of citric acid, sodium bicarbonate, sodium hydroxide, L-arginine and combinations thereof.

15. The powdered conditioner of claim 1, wherein the powdered condition has a pH of 3.5-4.5 when added at 10% to a water solution.

16. The powdered conditioner of claim 2, wherein the one or more carrier materials or flow agents comprise approximately 50% to approximately 90% by weight of the formulation.

17. The powdered conditioner of claim 2, wherein the one or more carrier materials or flow agents are selected from the group consisting of tapioca starch, corn starch, rice starch, silica, potato starch, arrowroot starch, tricalcium phosphate, powdered cellulose, magnesium stearate, sodium ferrocyanide, potassium ferrocyanide, calcium ferrocyanide, sodium silicate, silicon dioxide, calcium silicate, magnesium trisilicate, talcum powder, sodium aluminosilicate, potassium aluminum silicate, calcium aluminosilicate, bentonite, aluminum silicate, stearic acid, polydimethylsiloxane and combinations thereof.

18. The powdered conditioner of claim 1, further comprising one or more additives.

19. The powdered shampoo of claim 1, wherein the at least one additive in total comprises up to approximately 20% by weight of the formulation.

20. The powdered conditioner of claim 18, wherein the one or more additives comprise one or more preservatives, fragrances, thickening agents, or colorants.

21. A method comprising:
providing a powdered conditioner according to claim 1;
applying an amount of powdered conditioner to a user's hands;
rubbing the amount of powdered conditioner between the user's hands to form a liquid conditioner;
applying the liquid conditioner to the user's hair;
massaging the liquid conditioner into the user's hair; and
rinsing the liquid conditioner out of the user's hair.
